(19) United States
(12) Patent Application Publication
(10) Pub. No.: US 2024/0411048 A1
(43) Pub. Date: Dec. 12, 2024

(54) SYSTEM AND SELF-LEARNING METHOD FOR THE INTERPRETATION OF PETROPHYSICAL PARAMETERS

(71) Applicant: Abu Dhabi National Oil Company, Abu Dhabi (AE)

(72) Inventors: Abdul RAVOOF SHAIK, Abu Dhabi (AE); Ali ALSUMAITI, Abu Dhabi (AE); Budoor ALSHEHHI, Abu Dhabi (AE); Shehadeh MASALMEH, Abu Dhabi (AE); Ahmed BINAMRO, Abu Dhabi (AE)

(73) Assignee: Abu Dhabi National Oil Company, Abu Dhabi (AE)

(21) Appl. No.: 18/701,538

(22) PCT Filed: Apr. 1, 2022
(86) PCT No.: PCT/IB2022/053058

§ 371 Date: Apr. 15, 2024

(51) Int. Cl. G01V 20/00; G06F 30/27; G06F 30/28; G06F 113/08; G06F 119/14

(52) U.S. Cl. CPC G01V 20/00; G06F 30/27; G06F 30/28; G06F 2113/08; G06F 2119/14

(57) ABSTRACT

A computer-implemented method and system for approximating a predicted relative permeability curve and a predicted capillary pressure curve for a core plug sample are disclosed. The method comprises predicting the relative permeability curve and the capillary pressure curve for the core plug sample. The predicting is done using a trained machine learning algorithm and input measured pressure drop values, input measured fluid properties, input measured porous medium properties, and input measured fluid saturation profiles.

### FIELD OF THE INVENTION

The field of the invention relates to a system and method for modelling petrophysical parameters in a geological core analysis.

### BACKGROUND OF THE INVENTION

Modern field development of subsurface hydrocarbons relies on simulations of fluid and rock properties of the subsurface in oil and gas (hydrocarbon) reservoirs. Efficient field development is therefore dependent on reliable simulation of these fluid and rock properties. The oil and gas reservoirs typically contain water and oil or gas in varying amounts trapped between rock structures. Each of these components of the oil and gas reservoirs interferes with each other and impedes the flow of the other components in the hydrocarbon reservoir. The determination of a fluid saturation and modelling the fluid and rock properties in the hydrocarbon reservoir is therefore useful for the field development.

Various methods and systems for determining the fluid and rock properties are known in the prior art. Core data is typically obtained from samples derived from bore holes in order to determine the fluid and rock properties of the subsurface. Rock properties, such as porosity and permeability, are measured on core plugs cut from the core. A so called special core analysis (SCAL) is conducted on the core plugs and parameters describing fluid flow movements inside the core plugs are thereby obtained. Properties, such as relative permeability (Kr) and capillary pressure (Pc) during drainage and imbibition of the rock are determined from SCAL data produced during the SCAL analysis [1-3]. The capillary pressure is defined as the difference in pressure across the interface between two immiscible fluids, say oil and water, in the reservoir. Relative permeability can be defined as the ratio of the permeability to a given fluid in the presence of other fluids to the absolute permeability [4].

The SCAL analysis can further be used to determine, for example, a residual oil saturation in the presence of the water or the gas (in case of gas injection), electrical properties, compressibility, and stress dependency of petrophysical properties. Moreover, SCAL data can be used to evaluate an efficiency of enhanced oil recovery projects [5]. For example, low salinity water flooding (LSF) requires both high salinity and low salinity relative permeability and capillary pressure experiments for proper evaluation of each development option for the oil and gas reservoir [6, 7]. Interpretation of the SCAL data is a relevant technical discipline in the field development and helps to understand the fundamentals of rock and fluid interactions [8, 9].

SCAL therefore helps to provide relevant data for reservoir evaluation and for improving a production performance of the reservoir. This SCAL data is, for example, used for i) initializing and forecasting reservoir production performance in development, ii) understanding/estimating remaining oil saturation and oil distribution on both laboratory and reservoir scale, iii) assessing approaches to develop the field development (e.g., waterflooding, gas injection, or EOR), and iv) designing and optimizing injection schemes (e.g., in waterflooding and EOR processes).

As noted above, the SCAL data, such as the relative permeability and the capillary pressure, are useful in the field development. It is, however, challenging to generalize relative permeability curves and capillary pressure curves from core plug data to a field scale because both of the parameters (relative permeability and capillary pressure) are strongly dependent on both the rock and fluid properties and interactions between the rock and the fluid [10].

Several attempts have been made in the past to use artificial intelligence techniques to estimate relative permeability and capillary pressure. In document [11] it is proposed to predict relative permeability as a function of porosity, fluid viscosity, absolute permeability, water saturation, in combination with using Baker and Wyllie parameter combinations. However, in [11] the wettability effect on relative permeability is not considered which is an invalid assumption for oil reservoirs, both carbonate and sandstone. Document [12] describes a relative permeability model, wherein the model is a function of an Euler number, a pore geometry, a wettability, and a saturation history. Identifying the Euler number is a challenging and expensive task, as can be seen from Chinese patent document CN 112989653 A.

Most recently Document [9] used synthetic SCAL data to train an artificial intelligence model. In the approach disclosed by Matthew et. al. a steady state experimental data of a sample core plug is used as an input to a trained artificial intelligence model in order to predict the relative permeability and the capillary pressure. However, the proposed AI model does not have a self-learning capability and the AI model of [9] therefore needs to be trained for each core plug separately. This is an expensive and time-consuming task.

U.S. Pat. No. 7,333,892 B2 describes a system and method for determining multiphase flow parameter of a porous medium from an interpretation of displacement experiments. The multiphase flow is determined by identifying and calculating flow parameters, such as a relative permeability and a capillary pressure of a porous medium. A numerical flow simulator is applied to sections or volumes of the porous medium. A relative permeability profile and a capillary pressure profile are then determined using the flow simulator. The relative permeability profile and the capillary pressure profile are then used to evaluate a subsurface hydrocarbon accumulation.

International patent application WO 2020/231918 A1 describes a computer-implemented method and system for receiving test seismic data associated with a known truth interpretation and one or more hard constraints. The method further describes training a machine learning system based on the test seismic data, the known truth interpretation, and the one or more hard constraints. An error is then determined based on the training of the machine learning system and the system is adjusted based on the one or more soft constraints and the adjusted error data.

International patent application WO 2019/090316 A1 describes a computer-implemented method and system for determining a feasible region within a relative permeability and capillary pressure curve. The method comprises receiving downhole logging data for a porous medium. A pore size distribution index is estimated based upon nuclear magnetic resonance data (NMR) from a downhole logging data of the porous medium. A pore size distribution index of the porous medium is then determined. Curves for a relative permeability and a capillary pressure of the subsurface are then generated. The generation of the relative permeability and capillary pressure curves includes defining one or more of upper bound and lower bound relative permeability curves for oil and water, capillary pressures. Generating the relative permeability and capillary pressure curves further includes reducing the feasible region of solutions of the relative permeability and capillary pressure curves based on the defined upper and lower bound. The method discloses the selecting of relevant values for the relative permeability and the capillary pressure.

The prior art discloses solutions for determining relative permeability and the capillary pressure from the SCAL data. The solutions proposed in the prior art rely on analytical or mathematical determination of the relative permeability and the capillary pressure. These solutions do not consider effects of capillary forces at all or do only insufficiently reflect pore structure information of the porous medium. These calculations can also lead to unfeasible relative permeability or capillary pressure curves.

Measurements of the SCAL data are commonly only performed on a small group of samples. Artefacts of the SCAL measurements can, however, lead to unrealistic conclusions and thereby require additional processing of the SCAL data before use. Simulations of SCAL experiments are therefore often times used to expedite generating of the SCAL data and to accurately estimate further values for the relative permeability in order to determine the movable oil saturation in the porous medium [6].

### SUMMARY OF THE INVENTION

The present document describes a system and computer-implemented method for modelling petrophysical parameters such as a relative permeability curve and capillary pressure curve using a trained machine learning algorithm thereby reducing calculation times and improving a prediction accuracy for the relative permeability curve and the capillary pressure curve. A method for training of the machine learning algorithm is also disclosed.

The computer-implemented method for approximating the predicted relative permeability curve and the predicted capillary pressure curve for a core plug sample comprises predicting the relative permeability curve and the capillary pressure curve for the core plug sample. The prediction is done using a trained machine learning algorithm and input measured pressure drop values, input measured fluid properties, input measured porous medium properties, and input measured fluid saturation profiles.

The computer-implemented method further comprises generating simulated pressure drop values and simulated fluid saturation profiles. The generating is done using a fluid flow simulator and at least one of input relative permeability curve, input capillary pressure curve, input measured porous medium properties, and input measured fluid properties.

The method further comprises comparing the simulated pressure drop values and the simulated fluid saturation profiles with the measured pressure drop values and the measured fluid saturation profiles.

The computer-implemented method also comprises updating the trained machine learning algorithm using additional information such as a defined relative permeability curve, a defined capillary pressure curve, the measured fluid properties, the measured porous medium properties, the simulated pressure drop values, the simulated fluid saturation profiles, and a core flooding database.

The computer-implemented method may further comprise outputting, using an output unit, at least one of the predicted relative permeability curve and the predicted capillary pressure curve.

The use of the computer-implemented method may accelerate the estimation of capillary pressure curves and relative permeability curves while maintaining an acceptable accuracy. Moreover, the computer-implemented method is self-sufficient, i.e., it learns directly from the interaction with a fluid flow simulator, such as a reservoir simulator, without human intervention. Thereby efficiency may be increased.

Furthermore, by learning from the interaction with a fluid flow simulator the need for large amounts of data which are difficult to obtain is reduced. Even further, said method ensures that effects of capillary forces are taken into account, which may allow for an increased precision. Even further, said method allows to adequately reflect existing pore structure information, which may also allow for an increased precision.

The step of outputting may be performed, if the simulated pressure drop values and the simulated fluid saturation profiles are within an allowable level of the measured pressure drop values and the input measured fluid saturation profiles.

The outputted at least one of the predicted relative permeability curve and the predicted capillary pressure curve may be utilized to develop an oilfield. Hence, in view of the advantages described above, a more efficient and/or precise oilfield development can be achieved.

Thereby developing of an oilfield may comprise drilling at least one well-bore based on at least one of the predicted relative permeability curve and the predicted capillary pressure curve. Thereby the drilling location and/or depth may be determined precisely.

Further, developing of an oilfield may comprise controlling at least one parameter of a waterflooding process of the oilfield, a gas injection into the oilfield, and/or an enhanced oil recovery of the oilfield. Thereby the controlling of the at least one parameter may be based on at least one of the predicted relative permeability curve and the predicted capillary pressure curve. Hence, a more efficient development of the oilfield may be achieved. Particularly since resources required for waterflooding, gas injection and/or enhanced oil recovery may be utilized more efficiently.

The trained machine learning algorithm as mentioned in para. [0015] above may be trained by a computer-implemented method for training of the machine learning algorithm as described below starting from para. [0027].

Further, the updating may be performed, if the simulated pressure drop values and the simulated fluid saturation profiles are not within an allowable level of the measured pressure drop values and the input measured fluid saturation profiles.

A computer-implemented method for training of the machine learning algorithm comprises defining the relative permeability curve and the capillary pressure curve for a sample area in a reference core sample, wherein the sample area comprises porous medium and/or at least one fluid. The defined relative permeability curve and defined capillary pressure curve are generated using prior art mathematical models.

The computer-implemented method further comprises determining porous medium properties and fluid properties for the sample area using a detection device.

The computer-implemented method further comprises generating simulated pressure drop values and simulated fluid saturation profiles. The determining is done using a fluid flow simulator and inputting at least one of defined relative permeability curve, defined capillary pressure curve, determined porous medium properties, and determined fluid properties.

The computer-implemented method also comprises training the machine learning algorithm using the defined relative permeability curve, the defined capillary pressure curve, the measured porous medium properties, the determined fluid properties, the simulated pressure drop values, and the simulated fluid saturation profiles.

Since both above-described computer-implemented methods relate to a machine learning algorithm it is understood that any feature described in relation to the machine learning mechanism may apply for both computer-implemented methods. Particularly since the first computer-implemented method refers to a prediction which is done using a trained machine learning algorithm and the second computer-implemented method refers to a training of a machine learning algorithm the interrelation between said computer-implemented methods will be understood. Nevertheless, it is reemphasized that by learning from the interaction with a fluid flow simulator the need for large amounts of data which are difficult to obtain is reduced for the method for training of the machine learning algorithm.

A system for approximating a predicted relative permeability curve and a predicted capillary pressure curve is also disclosed. The system comprises an input unit, a storage unit, a processing unit, and an output unit. The input unit is used for inputting items of data obtained from a core plug sample using a detection device. The storage unit is used for storing items of input data. The processing unit is used for processing items of data related to at least one of a fluid flow simulator and a machine learning algorithm. The output unit is used for outputting items of data. Since the system serves for approximating a predicted relative permeability curve and a predicted capillary pressure curve it is understood that features and/or advantages which are described with regards to the computer-implemented method for approximating a predicted relative permeability curve and a predicted capillary pressure curve also apply for said system. Moreover, it is understood that the system may serve to perform said computer-implemented method

The system may further comprise means for predicting, using a trained machine learning algorithm and at least one of input measured pressure drop values, input measured fluid properties, input measured porous medium properties, and input measured fluid saturation profiles, the predicted relative permeability curve and the predicted capillary pressure curve. Further, the system may comprise means for generating, using a fluid flow simulator and at least one of an input predicted relative permeability curve, input predicted capillary pressure curve, input measured porous medium properties, and input measured fluid properties, simulated pressure drop values and simulated fluid saturation profiles. Moreover, the system may comprise means for comparing the simulated pressure drop values and the simulated fluid saturation profiles with the measured pressure drop values and the measured fluid saturation profiles. Furthermore, the system may comprise means for updating the machine learning algorithm using a defined relative permeability curve, a defined capillary pressure curve, the measured fluid properties, the measured porous medium properties, the simulated pressure drop values, the simulated fluid saturation profiles, and a core flooding database.

Further, a computer-readable medium is disclosed. Said computer-readable medium stores instructions that, when executed by a computer, cause it to perform any one of the above disclosed computer-implemented methods.

Further, a computer program is disclosed. Said computer program comprises instructions, which when executed by at least one processor cause the at least one processor to perform for performing any one of the above disclosed computer-implemented methods.

### DESCRIPTION OF THE FIGURES

FIG. 1 shows a flow chart describing the computer-implemented method for approximating a predicted relative permeability curve and a predicted capillary pressure curve for a core plug sample.

FIG. 2 shows a flow chart describing the computer-implemented method for training of a machine learning algorithm.

FIG. 3 shows an overview of a system for approximating a predicted relative permeability curve and a predicted capillary pressure curve for a core plug sample.

FIG. 4 shows a predicted relative permeability curve.

FIG. 5 shows a comparison of experimental and predicted water saturation profiles at different flow rates.

### DETAILED DESCRIPTION OF THE INVENTION

The invention will now be described on the basis of the figures. It will be understood that the embodiments and aspects of the invention described herein are only examples and do not limit the protective scope of the claims in any way. The invention is defined by the claims and their equivalents. It will be understood that features of one aspect or embodiment of the invention can be combined with a feature of a different aspect or aspects and/or embodiments of the invention.

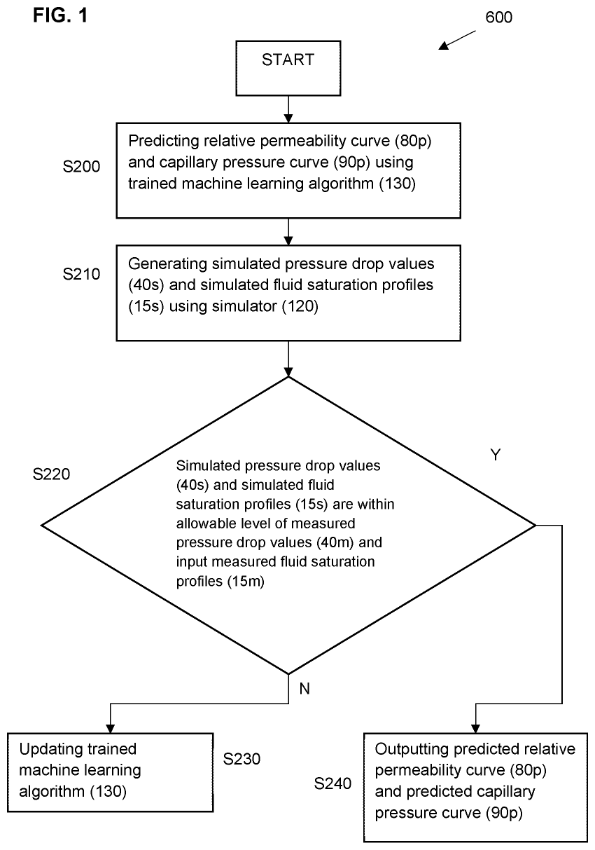

FIG. 1 shows a flow chart describing a computer-implemented method 600 for approximating a predicted relative permeability curve 80p and a predicted capillary pressure curve 90p for a core plug sample 50. The core plug sample 50 is a sample of rock obtained as a core plug from the porous medium or rocks of a subsurface oil and gas reservoir. Special core analysis laboratory (SCAL) data is obtained from the core plug sample 50 using special core analysis. The SCAL comprises multiple measurements and is directed at extending items of data obtained from measurements of dynamic petrophysical properties to situations more representative of the reservoir conditions. For example, typical laboratory setup such as core flooding, porous plate, and centrifuge is used for the obtaining of the items of data. This SCAL data is used to understand a fluid flow movement and behavior inside the core plug sample 50. The SCAL data includes, for example, the relative permeability curve and the capillary pressure curve for the core plug sample 50 which are calculated based on experimentally measured items of data for the core plug sample 50.

Fluid flow properties in the core plug sample 50 are determined using, for example, linear X-Ray core flooding equipment. This determining of the fluid flow properties includes capturing information on a one-dimensional phase saturation inside the porous rock medium.

The method 600 shown in FIG. 1 comprises predicting in step S200 a relative permeability curve 80p and a predicted capillary pressure curve 90p for the core plug sample 50. The prediction is done using a trained machine learning algorithm 130 and ones of input measured pressure drop values 40m, input measured fluid properties 30m, input measured porous medium properties 20m, and input measured fluid saturation profiles 15m. The measured fluid properties 30m are, for example, a fluid viscosity or a fluid density. The measured fluid properties 30m are determined using, for example, a viscometer equipment. The measured porous medium properties 20m are, for example, a porosity or a permeability of the core plug sample 50. These measured porous medium properties 20m are measured using laboratory equipment, for example, a permeameter. The measured pressure drop values 40m are pressure drops observed along the core plug sample 50 at each fractional rate using, for example, a linear x-ray. The measured pressure drop values 40m are determined in steady state core flooding experiments. Here the steady state core flooding equipment may be linear x-ray based which can capture the measured fluid saturation profiles 15m.

Simulated pressure drop values 40s and simulated fluid saturation profiles 15s are generated in step S210 using a fluid flow simulator 120 and ones of an input predicted relative permeability curve 80p, an input predicted capillary pressure curve 90p, input measured porous medium properties 20m, and input measured fluid properties 30m. The fluid flow simulator 120 can be any type of fluid flow simulator which is capable of processing fluid flow simulation through porous medium such as CMG-IMEX reservoir simulator.

The simulated pressure drop values 40s and the simulated fluid saturation profiles 15s are compared in step S220 with the measured pressure drop values 40m and the input measured fluid saturation profiles 15m. This comparing in step S220 is also described as “validation test”. If the simulated pressure drop values 40s and the simulated fluid saturation profiles 15s, compared to the measured pressure drop values 40m and the input measured fluid saturation profiles 15m, are within an experimental uncertainty, the validation test is passed. Typically, the experimental uncertainty is defined by an equipment used and is, for example, a value being less than 5%. The predicted relative permeability curve 80p and predicted capillary pressure curve 90p are output in step S240 using an output unit 160 if the validation test is passed.

If the simulated pressure drop values 40s and the simulated fluid saturation profiles 15s, compared to the measured pressure drop values 40m and the input measured fluid saturation profiles 15m, are not within the experimental uncertainty, the validation test fails. If the validation test has failed, the machine learning algorithm 130 is updated in step S230. The updating of the machine learning algorithm 130 is done by the fluid flow simulator 120 using a defined capillary pressure curve god, a defined relative permeability curve 80d, the measured fluid properties 30m, the measured porous medium properties 20m, the simulated pressure drop values 40s, and the simulated fluid saturation profiles 15. The defined relative permeability curve 80d and the defined capillary pressure curve 90d for the core plug sample 50 are calculated using mathematical methods such as but not limited to Brooks-Corey, LET, and modified Corey models that are available in literature to define the capillary pressure curve and relative permeability curve. For example, the defined relative permeability curve 80d can be generated by varying Corey's exponents n_wd and n_od, saturation end-points s_wc, and the fitting parameters c_wd, c_od, a_wd, a_od and b_d as described in the modified Corey model [6]. The updating, in step S230, comprises appending the measured fluid properties 30m, the measured porous medium properties 20m, the defined capillary pressure curve god, and the defined relative permeability curve 80d, simulated pressure drop values 40s and the simulated fluid saturation profiles 15 to a core flooding database 111a. The core flooding database 111a is a database for storing a plurality of items of data relating to, for example, the relative permeability (KR) and capillary pressure (Pc) of the core plug sample 50. This core flooding database 111a is used for retraining the machine learning algorithm 130 is based on items of data in the appended core flooding database 111a. The machine learning algorithm 130 is trained using, for example, supervised machine learning such as neural networks, gradient boosting, graph based neural networks, or ensemble-based models.

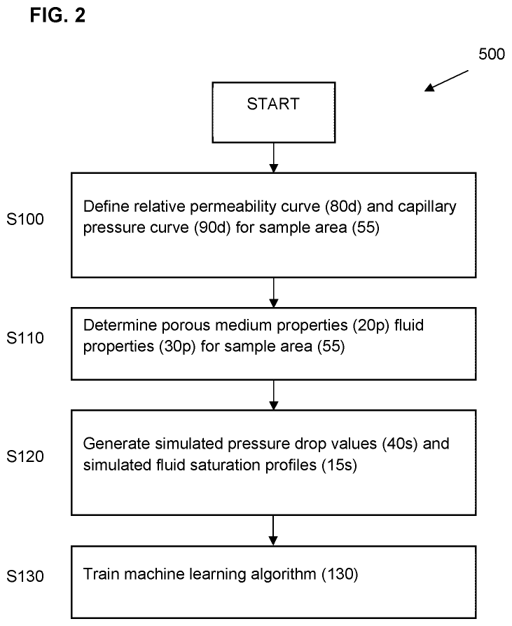

FIG. 2 shows a flow chart describing the computer-implemented method 500 for training of the machine learning algorithm 130. Based on the field observation, the relative permeability curve 80d and the capillary pressure curve god are defined for the sample area 55 in step S100. A range of synthetic fluid flow properties 30p and synthetic porous medium properties 20p are determined in step S110 for the sample area 55. These determined synthetic fluid flow properties 30p and these determined synthetic porous medium properties 20p are used to generate various scenarios in step S120. This methodology closely similar to the approach described in prior art (e.g. Mathew., et al 2021, Artificial Intelligence Coreflooding Simulator for Special Core Data Analysis). A fluid flow simulator 120 is used to generate the simulated pressure drop values 40s along the core plug, and the simulated fluid saturation profiles 15s along the core plugs for each scenario. The fluid flow simulator 120 can be any type of simulator which can handle multiphase fluid flow simulation through porous medium. For example, CMG-IMEX reservoir simulator is used in this work. Fluid flow properties 30p, porous medium properties 20p, simulated pressure drop values 40s and simulated fluid saturation profiles 15 are treated as input parameters while the defined capillary pressure curve god and relative permeability curve 80d as output parameters to train the machine learning algorithm 130. The machine learning algorithm 130 is then trained in step S130 using the core flooding database 111a which consists of the defined relative permeability curve 80d, the defined capillary pressure curve god, fluid flow properties, porous medium properties, the simulated pressure drop values 40s, and the simulated fluid saturation profiles 15s.

Prior art techniques are used for the purpose of training of the machine learning algorithm 130. The core flooding database 111a may be divided into two subsets as training subset and testing subset. Then, a predetermined accuracy threshold may be used to determine whether the training is successful as would be understood by one of ordinary skill in the art. A system 700 may compare the predicted relative permeability curve 80p and the predicted capillary pressure curve 90p with the defined relative permeability curve 80d, the defined capillary pressure curve god. Then, the system 700 may compare an accuracy value (e.g., percentage difference, difference, RMSE or MSE) with the predetermined accuracy threshold to determine whether the training is successful. The machine learning algorithm 130 can be a supervised machine learning technique based on but not limited to the neural network, gradient boosting, graph based neural network or ensemble-based model. In this work, extreme gradient boosting approach is used to train the machine learning algorithm 130.

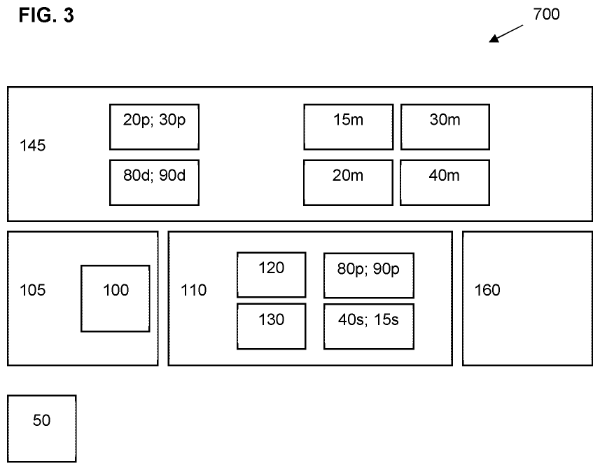

FIG. 3 shows an overview of the system 700 for approximating the predicted relative permeability curve 80p and the predicted capillary pressure curve 90p for the core plug sample 50. The system 700 comprises an input unit 105, a storage unit 145, a processing unit 11o, and the output unit 160. The input unit 105 is used for inputting items of data obtained from the core plug sample 50 and the reference core plug sample 50r using a detection device 100. The input items of data comprise the determined porous medium properties 20p, the determined fluid properties 30p, the defined relative permeability curve 80d, the defined capillary pressure curve god. The input items of data further comprise the measured fluid saturation profiles 15m, the measured porous medium properties 20m, the measured fluid properties 30m, and the measured pressure drop values 40m. The storage unit 145 is, for example, a read-only-memory ROM or a random-access-memory RAM. The storage unit 145 is used for storing the input items of data. The processing unit 110 is used for processing the input items of data related to at least one of the fluid flow simulator 120 or the machine learning algorithm 130. The output unit 160 is used for outputting the predicted relative permeability curve 80p and the predicted capillary pressure curve 90p.

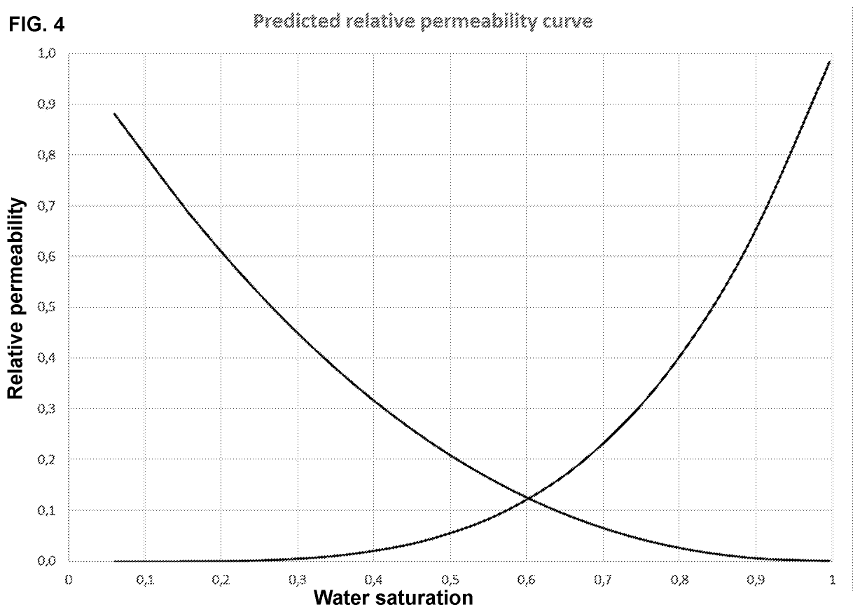

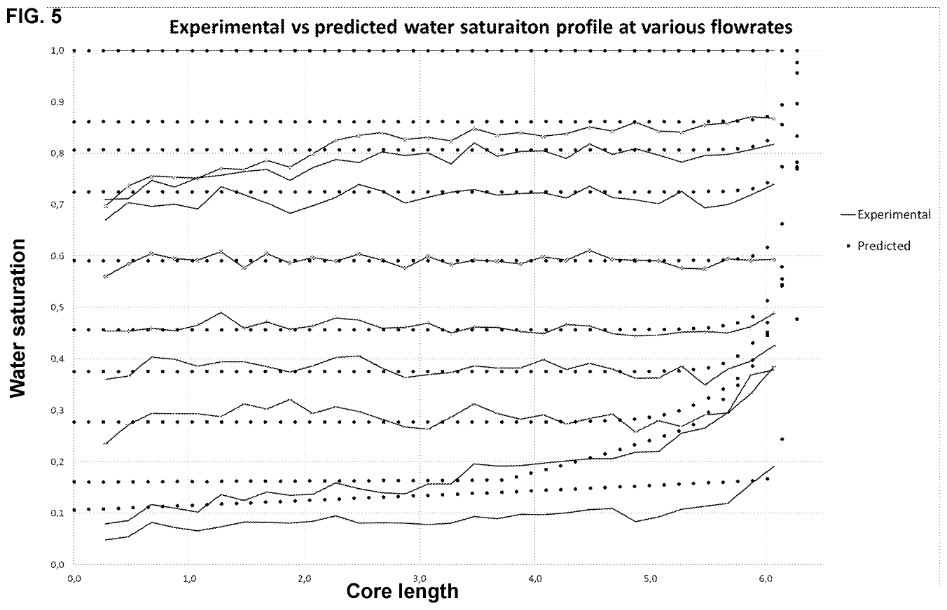

FIG. 4 depicts a typical example of the predicted relative permeability curve 80d of the core plug sample 50, whereas FIG. 5 shows a simulated water saturation along a length of the core plug sample 50 at each fractional flow (solid line) and a measured water saturation along the length of the core plug sample 50 at each fractional flow (dotted line). As can be seen from FIG. 5, the simulated water saturation along the length of the core plug sample 50 is closely matching with the measured water saturation.

### REFERENCES

1. Andersen, P. O., et al., Simulation Interpretation of Capillary Pressure and Relative Permeability From Laboratory Waterflooding Experiments in Preferentially Oil-Wet Porous Media. SPE Reservoir Evaluation & Engineering, 2020. 23(01): p. 230-246.
2. Al-Gharbi, M. S., et al. SCAL Relative Permeability Measurements and Analyses for a Cluster of Fields in South Oman. in International Petroleum Technology Conference. 2007.
3. Sorop, T. G., et al. Relative Permeability Measurements to Quantify the Low Salinity Flooding Effect at Field Scale. in Abu Dhabi International Petroleum Exhibition and Conference. 2015.
4. Baker, R. O., H. W. Yarranton, and J. L. Jensen, 8—Special Core Analysis-Rock-Fluid Interactions, in Practical Reservoir Engineering and Characterization, R. O. Baker, H. W. Yarranton, and J. L. Jensen, Editors. 2015, Gulf Professional Publishing: Boston. p. 239-295.
5. Masalmeh, S. K., et al. EOR Options for Heterogeneous Carbonate Reservoirs Currently Under Waterflooding. in Abu Dhabi International Petroleum Exhibition and Conference. 2014.
6. Masalmeh, S. K., I. M. Abu-Shiekah, and X. Jing, Improved Characterization and Modeling of Capillary Transition Zones in Carbonate Reservoirs. SPE Reservoir Evaluation & Engineering, 2007. 10(02): p. 191-204.
7. Nasralla, R. A., et al. Demonstrating the Potential of Low-Salinity Waterflood to Improve Oil Recovery in Carbonate Reservoirs by Qualitative Coreflood. in Abu Dhabi International Petroleum Exhibition and Conference. 2014.
8. Masalmeh, S. K., et al. Low Salinity Flooding: Experimental Evaluation and Numerical Interpretation, in International Petroleum Technology Conference. 2014.
9. Mathew, E. S., et al., Artificial Intelligence Coreflooding Simulator for Special Core Data Analysis. SPE Reservoir Evaluation & Engineering, 2021: p. 1-29.
10. Siddiqui, S., et al., Improvements in the Selection Criteria for the Representative Special Core Analysis Samples. SPE Reservoir Evaluation & Engineering, 2006. 9(06): p. 647-653.
11. Arigbe, O. D., et al., Real-time relative permeability prediction using deep learning. Journal of Petroleum Exploration and Production Technology, 2019. 9(2): p. 1271-1284.
12. Zhao, B., et al., A Hybrid Approach for the Prediction of Relative Permeability Using Machine Learning of Experimental and Numerical Proxy SCAL Data. SPE Journal, 2020. 25(05): P. 2749-2764.

### REFERENCE NUMERALS

15m measured fluid saturation profiles
15s simulated fluid saturation profiles
20p porous medium properties
20m measured porous medium properties
30m measured fluid properties
30p determined fluid properties
40m measured pressure drop values
40s simulated pressure drop values
50 core plug sample
50r reference core plug sample
55 sample area
80d defined relative permeability curve
80p predicted relative permeability curve
90d defined capillary pressure curve
90p predicted capillary pressure curve
100 detection device
105 input unit
110 processing unit
111a core flooding database
120 fluid flow simulator
130 machine learning algorithm
145 storage unit
160 output unit
500 method
600 method
700 system

1. A computer-implemented method for approximating a predicted relative permeability curve and a predicted capillary pressure curve for a core plug sample, the method comprising:
predicting, using a trained machine learning algorithm and at least one of input measured pressure drop values, input measured fluid properties, input measured porous medium properties, and input measured fluid saturation profiles, the predicted relative permeability curve and the predicted capillary pressure curve;
generating, using a fluid flow simulator and at least one of an input predicted relative permeability curve, input predicted capillary pressure curve, input measured porous medium properties, and input measured fluid properties, simulated pressure drop values and simulated fluid saturation profiles;
comparing the simulated pressure drop values and the simulated fluid saturation profiles with the measured pressure drop values and the measured fluid saturation profiles; and
updating the machine learning algorithm using a defined relative permeability curve, a defined capillary pressure curve, the measured fluid properties, the measured porous medium properties, the simulated pressure drop values, the simulated fluid saturation profiles, and a core flooding database.

2. The computer-implemented method of claim 1 further comprising:
outputting, using an output unit, at least one of the predicted relative permeability curve and the predicted capillary pressure curve.

3. The computer-implemented method of claim 1, wherein outputting is performed, if the simulated pressure drop values and the simulated fluid saturation profiles are within an allowable level of the measured pressure drop values and the input measured fluid saturation profiles.

4. The computer-implemented method according to claim 2, wherein the outputted at least one of the predicted relative permeability curve and the predicted capillary pressure curve are utilized to develop an oilfield.

5. The computer-implemented method according to claim 4, wherein developing of an oilfield comprises drilling at least one well-bore based on at least one of the predicted relative permeability curve and the predicted capillary pressure curve.

6. The computer-implemented method according to claim 4, wherein developing of an oilfield comprises controlling at least one parameter of a waterflooding process of the oilfield, a gas injection into the oilfield, and/or an enhanced oil recovery of the oilfield, wherein the controlling of the at least one parameter is based on at least one of the predicted relative permeability curve and the predicted capillary pressure curve.

7. The computer-implemented method of claim 1, wherein updating is performed, if the simulated pressure drop values and the simulated fluid saturation profiles are not within an allowable level of the measured pressure drop values and the input measured fluid saturation profiles.

8. The computer-implemented method of claim 1, wherein the trained machine learning algorithm is trained by a computer-implemented method.

9. A computer-implemented method for training of a machine learning algorithm, the method comprising:
defining a relative permeability curve and a capillary pressure curve for a sample area in a reference core sample, wherein the sample area comprises at least one of a porous medium and at least one fluid;
determining, using a detection device, porous medium properties and fluid properties for the sample area;
generating, using a fluid flow simulator and inputting at least one of defined relative permeability curve, defined capillary pressure curve, determined porous medium properties, and determined fluid properties, simulated pressure drop values and simulated fluid saturation profiles; and
training the machine learning algorithm using the defined relative permeability curves, the defined capillary pressure curve, the measured porous medium properties, the determined fluid properties, the simulated pressure drop values, and the simulated fluid saturation profiles.

10. A system for approximating a predicted relative permeability curve and a predicted capillary pressure curve, the system comprising:
an input unit for inputting items of data obtained from a core plug sample and a reference core plug sample using a detection device;
a storage unit for storing input items of data;
a processing unit for processing items of data related to at least one of a fluid flow simulator and a machine learning algorithm; and
an output unit for outputting items of data.

11. The system according to claim 10, wherein the system further comprises at least one of
means for predicting, using a trained machine learning algorithm and at least one of input measured pressure drop values, input measured fluid properties, input measured porous medium properties, and input measured fluid saturation profiles, the predicted relative permeability curve and the predicted capillary pressure curve;
means for generating, using a fluid flow simulator and at least one of an input predicted relative permeability curve, input predicted capillary pressure curve, input measured porous medium properties, and input measured fluid properties, simulated pressure drop values and simulated fluid saturation profiles;
means for comparing the simulated pressure drop values and the simulated fluid saturation profiles with the measured pressure drop values (40m) and the measured fluid saturation profiles; and
means for updating the machine learning algorithm using a defined relative permeability curve, a defined capillary pressure curve, the measured fluid properties, the measured porous medium properties, the simulated pressure drop values, the simulated fluid saturation profiles, and a core flooding database.

12. A computer-readable medium storing instructions that, when executed by a computer, cause it to perform a method according to claim 1.

13. A computer program, comprising instructions, which when executed by at least one processor cause the at least one processor to perform for performing a method, according to claim 1.
